(19) United States
(12) Patent Application Publication
(10) Pub. No.: US 2026/0021255 A1
(43) Pub. Date: Jan. 22, 2026

(54) HYSTEROSCOPY INJECTION NEEDLE

(71) Applicant: Rush University Medical Center, Chicago, IL (US)

(72) Inventors: Mary WOOD MOLO, Chicago, IL (US); Jessica Dai, Chicago, IL (US); Azra Sadikovic-Letko, Chicago, IL (US)

(73) Assignee: Rush University Medical Center, Chicago, IL (US)

(21) Appl. No.: 19/341,439

(22) Filed: Sep. 26, 2025

Related U.S. Application Data
(63) Continuation of application No. PCT/US2024/021996, filed on Mar. 28, 2024.
(60) Provisional application No. 63/455,006, filed on Mar. 28, 2023.

(51) Int. Cl. A61M 5/32; A61M 39/12

(52) U.S. Cl. CPC A61M 5/32; A61M 39/12; A61M 2202/0007; A61M 2210/1433

(57) ABSTRACT

The disclosure is directed to devices, systems, and methods for a hysteroscopy injection needle. One device for injecting a fluid into a tissue includes a shaft having an elongated lumen configured to deliver a fluid from a proximal end of the shaft to a distal end of the shaft; a handle fixed with the shaft, the handle configured to maneuver the distal end of the shaft at a tissue; and a connector at the proximal end of the shaft, the connector configured to receive a liquid handling device.

### RELATED APPLICATIONS

This application is a continuation application of PCT International Patent Application No. PCT/US2024/021996, filed on Mar. 28, 2024, which is herein incorporated by reference in its entirety. PCT International Patent Application No. PCT/US2024/021996 is based on and claims the benefit of priority to U.S. Provisional Application No. 63/455,006 filed on Mar. 28, 2023, which is herein incorporated by reference in its entirety.

### FIELD OF THE TECHNOLOGY

The present disclosure relates to apparatus and systems for injection needles for various endoscopes, particularly for hysteroscopy injection needles.

### BACKGROUND

An operative hysteroscope may be used during some medical diagnostics and/or treatments, for example, to achieve substantial tissue resection. However, a visual field of the hysteroscope may be obscured by bleeding of some body fluid from surrounding tissues, which can significantly limit a health care provider's ability to perform the medical diagnostics and/or treatments safely, resulting in longer operation duration and/or serious complications.

The present disclosure describes various embodiments for a hysteroscopy injection needle, addressing at least one of the issues/problems discussed above, thus, improving operating conditions with fewer interruptions and accomplishing the same result in less time, which may reduce a number of additional procedures needed, subsequent exposures to anesthesia, and/or associated health care costs of hysteroscopic procedures.

### SUMMARY

The present disclosure relates to devices, systems, and methods for a hysteroscopy injection needle.

In one embodiment, the present disclosure describes a device for injecting a fluid into a tissue, the device comprising a shaft having an elongated lumen configured to deliver a fluid from a proximal end of the shaft to a distal end of the shaft; a handle fixed with the shaft, the handle configured to maneuver the distal end of the shaft at a tissue; and a connector at the proximal end of the shaft, the connector configured to receive a liquid handling device. The shaft is thinner and longer than a sheath of a hysteroscope, and the shaft is configured to feed through the sheath of the hysteroscope. The sheath of the hysteroscope comprises an operative channel; and an outer diameter of the shaft is smaller than an inner diameter of the operative channel, and the shaft is configured to feed through the operative channel of the hysteroscope. The shaft is longer than the sheath of the hysteroscope along a longitudinal axis, so that when the shaft feeds through the sheath, the distal end of the shaft extends outside the sheath distally and the proximal end of the shaft extends outside the sheath proximally. The tissue comprises a fibroid; and the liquid comprises a vasoconstrictor reagent. The vasoconstrictor reagent comprises vasopressin.

The distal end of the shaft comprises a beveled end configured to pierce an outer layer of the tissue. The shaft comprises a rigid material with an outer diameter between 0.9 mm and 2.2 mm, inclusive. The handle is configured to be operated by a health care provider, so as to maneuver the distal end of the shaft to pierce the outer layer of the tissue to deliver the liquid into the tissue.

The connector at the proximal end of the shaft comprises a female luer lock connector. The liquid handling device comprises a syringe, wherein the female luer lock connector at the proximal end of the shaft is configured to connect to a male luer lock connector of the syringe. The liquid handling device comprises a flexible tube and a syringe, wherein: the female luer lock connector at the proximal end of the shaft is configured to connect to a male luer lock connector at a distal end of the flexible tube, and a proximal end of the flexible tube is configured to connect to the syringe. The proximal end of the flexible tube comprises a female luer lock connector; and the female luer lock connector at the proximal end of the flexible tube is configured to connect to a male luer lock connector of the syringe. The liquid handling device comprises a stopcock valve.

In some other embodiments, a system may include the device described above.

In some other embodiments, a method may include injecting a fluid into a tissue by using the device described above.

In some other embodiments, a method may include injecting a fluid into a tissue by using the system described above.

In some other embodiments, a method may include manufacturing the device described above.

In some other embodiments, a method may include manufacturing the system described above.

### BRIEF DESCRIPTION OF THE DRAWINGS

The system, device, product, and/or method described below may be better understood with reference to the following drawings and description of non-limiting and non-exhaustive embodiments. The components in the drawings are not necessarily to scale. Emphasis instead is placed upon illustrating the principles of the disclosure.

FIG. 1 shows a schematic diagram of one exemplary embodiment in the present disclosure.

FIG. 2A shows a schematic diagram of one exemplary embodiment without any attachment.

FIG. 2B shows a schematic diagram of one exemplary embodiment with a syringe with luer lock tip attached.

FIG. 2C shows a schematic diagram of one exemplary embodiment with accessory tubing and a syringe with luer lock tip attached.

FIG. 3 shows a schematic diagram of a hysteroscope in the present disclosure.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the embodiments above and the claims below. Reference should therefore be made to the embodiments above and claims below for interpreting the scope of the invention.

### DESCRIPTION OF THE DISCLOSURE

The device and methods now will be described more fully hereinafter with reference to the accompanying drawings, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements.

Likewise, many modifications and other embodiments of the device and methods described herein will come to mind to one of skill in the art to which the invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

Throughout the specification and claims, terms may have nuanced meanings suggested or implied in context beyond an explicitly stated meaning. Likewise, the phrase “in one embodiment” or “in one implementation” as used herein does not necessarily refer to the same embodiment or implementation and the phrase “in another embodiment” or “in another implementation” as used herein does not necessarily refer to a different embodiment or implementation. It is intended, for example, that claimed subject matter includes combinations of exemplary embodiments or implementations in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” or “at least one” as used herein, depending at least in part upon context, may be used to describe any feature, structure, or characteristic in a singular sense or may be used to describe combinations of features, structures or characteristics in a plural sense. Similarly, terms, such as “a”, “an”, or “the”, again, may be understood to convey a singular usage or to convey a plural usage, depending at least in part upon context. In addition, the term “based on” or “determined by” may be understood as not necessarily intended to convey an exclusive set of factors and may, instead, allow for existence of additional factors not necessarily expressly described, again, depending at least in part on context.

Unless defined otherwise, all technical and scientific terms used herein have the same meaning as commonly understood by one of skill in the art to which the invention pertains. Although any methods and materials similar to or equivalent to those described herein may be used in the practice or testing of the present invention, the preferred methods and materials are described herein.

The present disclosure describes various embodiments for devices, apparatus, and/or systems of injection needles for various endoscopes, particularly for hysteroscopy injection needles. The present disclosure includes various embodiments for injecting a fluid into a tissue by using the devices, apparatus, and/or systems described herein. The present disclosure also includes various embodiments for manufacturing the devices, apparatus, and/or systems described herein.

The present disclosure describes hysteroscope as an example of various endoscopes, which does not impose limitation on the applicability and scope of the described embodiments.

In some implementations, fibroids affect a majority of women during their reproductive years and are a common cause of abnormal uterine bleeding. While open and laparoscopic surgeries have their own respective needles to deliver vasopressin to decrease blood loss, there is no equivalent in hysteroscopy, a surgery reliant on a small visual field.

In some implementations, challenging hysteroscopic myomectomies are reliant on skillful and efficient use of the operative hysteroscope in order to achieve substantial tissue resection prior to meeting the fluid deficit. However, skill alone is not always sufficient to complete a resection. Because of the rich vascularity of fibroids, the visual field can easily become obscured by bleeding into the distention fluid. Bleeding can limit the surgeon's ability to operate safely and result in the need for multiple surgeries to successfully resect a fibroid.

The present disclosure describes various embodiment for providing a solution to a common but prohibitive problem in the field of hysteroscopy. When the surgeon is able to control bleeding and sustain their visualization during surgery, they will be better equipped to operate with fewer interruptions and accomplishing the same result in less time. In turn, this will reduce the number of additional procedures needed, subsequent exposures to anesthesia, and associated health care costs of hysteroscopic myomectomies.

In some implementations with abdominal and laparoscopic myomectomies, a vasoconstrictor such as vasopressin may be administered to decrease bleeding. The present disclosure describes various embodiments for a hysteroscopic injection needle, which may decrease bleeding and improve visualization during hysteroscopic myomectomies. In some implementations, the needle may be designed to deliver vasopressin to a submucosal fibroid under direct hysteroscopic visualization, wherein the needle may be inserted through a straight operative channel of a rigid hysteroscope.

Various embodiments in the present disclosure may have the following objectives and/or commercial applications: allowing for easy intracavitary administration of vasopressin to a submucosal fibroid, and/or decreasing the blood loss during hysteroscopic resection. With less bleeding into the distension fluid, the surgeon will be able to maintain clearer and safer visualization of the uterine cavity.

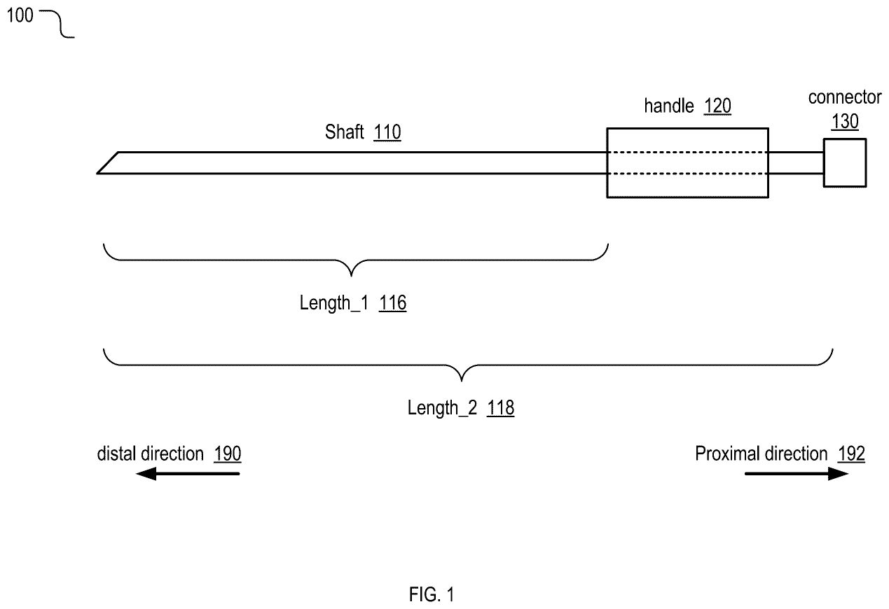

FIG. 1 shows an exemplary embodiment of an injection needle 100. The injection needle may include a portion or all of the following: a shaft 110, a handle (or grip) 120, and/or a connector 130.

In the present disclosure, a distal end (or direction) 190 may refer to an end (or direction) away from a health care provider (e.g., a physician, a surgeon doctor, or an assistant to a physician/surgeon) who operates the injection needle, and a proximal end (or direction) 192 may refer to an end (or direction) towards the health care provider who operates the injection needle.

In some implementations, the shaft may be made of metal, and have a certain rigidity so that when the health care provider operates the handle, a distal end of the shaft may be maneuvered to penetrate an outer layer of a target tissue relatively easily with a certain precision. For example, the target tissue may be one or more fibroids in the hysteroscopic occasions.

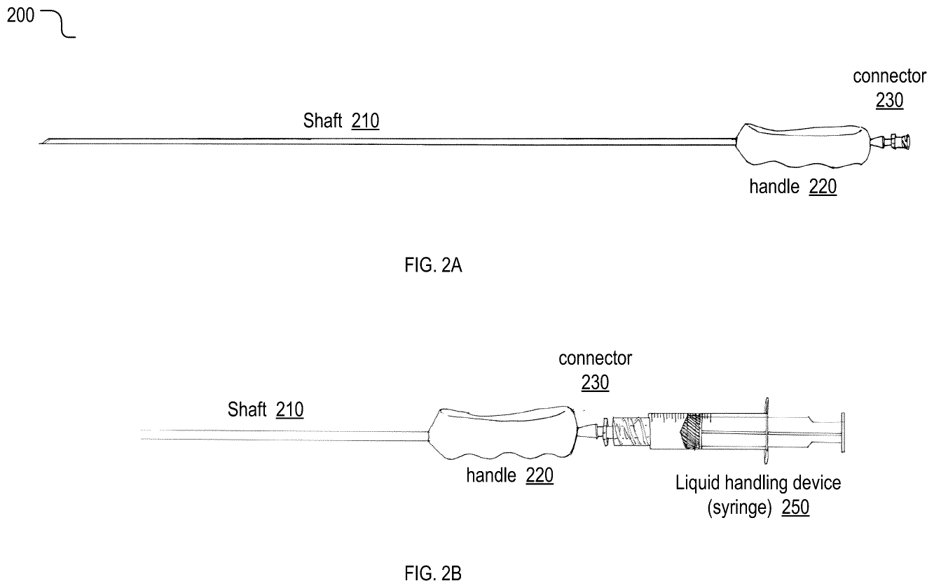

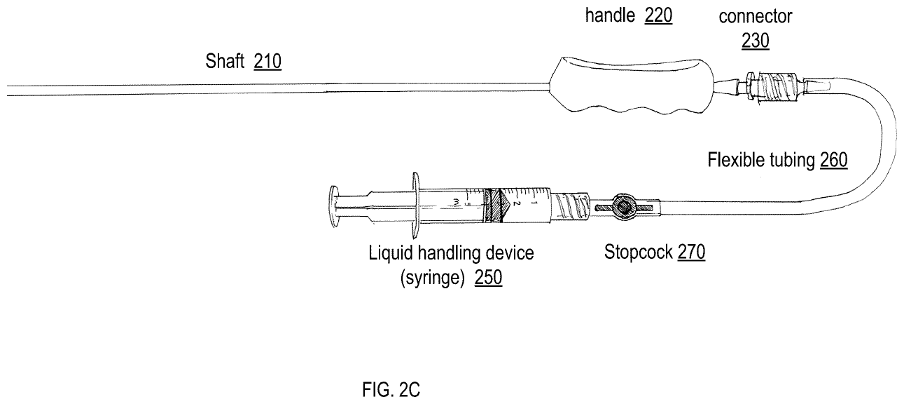

FIG. 2A shows another schematic drawing of an exemplary embodiment of an injection needle 200. The injection needle may include a portion or all of the following: a shaft 210, a handle (or grip) 220, and/or a connector 230.

In some implementations wherein the injection needle is used for injecting a fluid into a tissue, optionally or alternatively in combination with one or more implementations and/or embodiments described in the present disclosure, the shaft may have an elongated lumen configured to deliver a fluid from a proximal end of the shaft to a distal end of the shaft; the handle fixed with the shaft, the handle configured to maneuver the distal end of the shaft at a tissue; and/or the connector at the proximal end of the shaft, the connector configured to receive a liquid handling device. In some implementations, the handle may be made of medical grade plastic.

In some implementations, optionally or alternatively in combination with one or more implementations and/or embodiments described in the present disclosure, the shaft is thinner and longer than a sheath of a hysteroscope, and the shaft is configured to feed through the sheath of the hysteroscope.

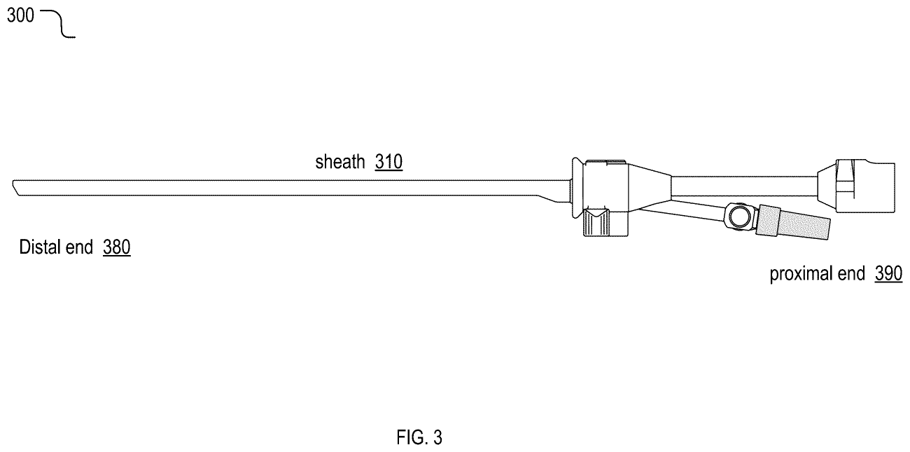

FIG. 3 shows a schematic drawing of an exemplary hysteroscope 300, which including a sheath 310 has a distal end 380 and a proximal end 390. The hysteroscope has a longitudinal axis along distal-proximal direction. In some implementations, the sheath includes a portion or all of the following channels, an inflow channel, an outflow channel, an optical channel carrying optical fibers, an operative channel for introducing scissors, graspers, and/or biopsy instruments. For non-limiting examples, hysteroscopes may include 6.25 mm or 7.25 mm operative hysteroscopes.

In some implementations, optionally or alternatively in combination with one or more implementations and/or embodiments described in the present disclosure, the sheath of the hysteroscope comprises an operative channel; and an outer diameter of the shaft is smaller than an inner diameter of the operative channel, and the shaft is configured to feed through the operative channel of the hysteroscope.

In some implementations, optionally or alternatively in combination with one or more implementations and/or embodiments described in the present disclosure, the shaft is longer than the sheath of the hysteroscope along a longitudinal axis, so that when the shaft feeds through the sheath, the distal end of the shaft extends outside the sheath distally and the proximal end of the shaft extends outside the sheath proximally.

In some implementations, the length of the needle shaft needs to extend beyond the distal end of the operating channel of the hysteroscope to allow proper visualization of the bevel during tissue penetration. The shaft needs to fit for the hysteroscope and maintain the rigidity needed to penetrate fibroids.

In some implementations, the needle may have a total length (length_2 118 in FIG. 1), measuring from the distal end to the proximal end of the shaft. In some implementation, the needle may have a length (length_1 116 in FIG. 1), which need to be longer than the sheath of the hysteroscope, so that the distal end of shaft extends beyond the distal end of the sheath while the handle maintains outside the sheath of the hysteroscope for operation. In some implementations, the length of the needle may be in a range between 30 centimeter (cm) and 40 cm, inclusive.

In some implementations, optionally or alternatively in combination with one or more implementations and/or embodiments described in the present disclosure, the tissue comprises a fibroid; and/or the liquid comprises a vasoconstrictor reagent.

In some implementations, optionally or alternatively in combination with one or more implementations and/or embodiments described in the present disclosure, the vasoconstrictor reagent comprises vasopressin.

In some implementations, optionally or alternatively in combination with one or more implementations and/or embodiments described in the present disclosure, the distal end of the shaft comprises a beveled end configured to pierce an outer layer of the tissue.

In some implementations, optionally or alternatively in combination with one or more implementations and/or embodiments described in the present disclosure, the shaft comprises a rigid material with an outer diameter between 0.9 mm and 2.2 mm, inclusive. For non-limiting examples, the shaft may be similar or same as a 16 Gauge needle with an outer diameter of about 1.65 millimeter (mm), a 18 Gauge needle with an outer diameter of about 1.27 mm, a 14 Gauge needle with an outer diameter of about 2.1 mm, or a 20 Gauge needle with an outer diameter of about 0.91 mm. In the present disclosure, “about A” may refer to a range from 95% A to 105% A, inclusive.

In some implementations, optionally or alternatively in combination with one or more implementations and/or embodiments described in the present disclosure, the handle is configured to be operated by a health care provider, so as to maneuver the distal end of the shaft to pierce the outer layer of the tissue to deliver the liquid into the tissue.

FIG. 2B shows a schematic diagram of one exemplary needle connecting with a syringe 250. FIG. 2C shows a schematic diagram of one exemplary needle with flexible tubing 260 and a syringe 250. In some implementations, the flexible tubing may include a stopcock valve 270.

In some implementations, the needle grip or handle is located at the proximal end of the device and is designed to allow for small and precise movements. The needle shaft traverses the length of the grip and remains patent, connecting the needle lumen to the connector (e.g., a female luer lock cap) at the end of the grip, allowing for: (1) direct attachment of a syringe (with a luer lock); or (2) attachment of a specially designed and flexible tubing extension.

In some implementations, the connector of the needle may include a luer type connector (e.g., a female luer lock cap, a male luer lock cap, a female luer slip connector, or a male luer slip connector), a fitting type connector (e.g., an instant fitting), or a coupling type connector (e.g., a quick coupling). In some implementations, any one of the above types of connectors may include a shutoff valve or may be added with a shutoff valve.

In some implementations, optionally or alternatively in combination with one or more implementations and/or embodiments described in the present disclosure, the connector at the proximal end of the shaft comprises a female luer lock connector.

In some implementations, optionally or alternatively in combination with one or more implementations and/or embodiments described in the present disclosure, the liquid handling device comprises a syringe, wherein the female luer lock connector at the proximal end of the shaft is configured to connect to a male luer lock connector of the syringe.

In some implementations, optionally or alternatively in combination with one or more implementations and/or embodiments described in the present disclosure, the liquid handling device comprises a flexible tube and a syringe, wherein: the female luer lock connector at the proximal end of the shaft is configured to connect to a male luer lock connector at a distal end of the flexible tube, and/or a proximal end of the flexible tube is configured to connect to the syringe.

In some implementations, optionally or alternatively in combination with one or more implementations and/or embodiments described in the present disclosure, the proximal end of the flexible tube comprises a female luer lock connector; and/or the female luer lock connector at the proximal end of the flexible tube is configured to connect to a male luer lock connector of the syringe.

In some implementations, optionally or alternatively in combination with one or more implementations and/or embodiments described in the present disclosure, the liquid handling device comprises a stopcock valve. The stopcock allows a precise control of delivering the liquid from the liquid handling device to the tissue. In the hysteroscopic procedure, a certain amount of diluted vasopressin fluid may be delivered to one or more fibroids, and the amount may depend on a portion or all of the following factors: the surgeon, a number of fibroids, sizes of the fibroids, and/or vasopressin concentration. For non-limiting examples, a syringe attached may be a 5 milliliter (mL or cc) or 10 cc syringe, which may be refilled/used on more than one occasions, (e.g., when more vasopressin is needed).

In some implementations, the direct syringe attachment is most suitable for a surgeon operating alone. The aforementioned tubing extension has a male luer lock on the distal end and a stop-cock with a female luer lock cap on the proximal end that attaches to the syringe. In some implementations, this extension allows for a surgical assistant to aid in the administration of the vasopressin while the surgeon holds the hysteroscope and needle in place.

In various embodiments, a system may include a device according to any combination of one or more implementations/embodiments described in the present disclosure.

Various embodiments may include steps of injecting a fluid into a tissue by using a device according to any combination of one or more implementations/embodiments described in the present disclosure.

Various embodiments may include steps of injecting a fluid into a tissue by using a system according to any combination of one or more implementations/embodiments described in the present disclosure.

Various embodiments may include steps of manufacturing a device according to any combination of one or more implementations/embodiments described in the present disclosure.

Various embodiments may include steps of manufacturing a system according to any combination of one or more implementations/embodiments described in the present disclosure.

Hysteroscopy is one of the most common and least invasive surgeries that a gynecologist performs. In some implementations, despite the widespread use of hysteroscopy for the management of submucosal fibroids, there is no such device on the market. The present disclosure describes various embodiment for hysteroscopy, serving as the only means for administering intra-uterine vasopressin via a hysteroscope. Various embodiments in the present disclosure has the potential to amplify the effectiveness of operative hysteroscopy, and/or have the potential to become a regular staple in the inventory of the operating rooms for gynecological surgery.

The present disclosure includes methods for injecting a fluid into a tissue by using the devices, apparatus, and/or systems described herein. One method may include a portion or all of the following (not necessarily with the order as described below): feeding a shaft of an injection needle through a sheath of a hysteroscope, connecting a syringe to a proximal end of the injecting needle, operating a handle of the injection needle to maneuver a distal end of the needle to penetrate a fibroid, and/or delivering an amount of liquid from the syringe into the fibroid. The method does not necessary include the exact order as described above, and a persons skilled in the art may understand that, based on the present disclosure, another method may include a different order.

The present disclosure also includes methods for manufacturing the devices, apparatus, and/or systems described herein. One method may include a portion or all of the following (not necessarily with the order as described below): fabricating a shaft of an injection needle, disposing a handle onto a proximal portion of the shaft, and/or disposing a connector at a proximal end of the shaft, wherein the shaft has a beveled (sharp) tip at a distal end. The method does not necessary include the exact order as described above, and a persons skilled in the art may understand that, based on the present disclosure, another method may include a different order.

Through the descriptions of the preceding embodiments, persons skilled in the art may understand that the device, system, and/or method according to the foregoing embodiments may be implemented by hardware only or by software and a necessary universal hardware platform. In some cases, using software and a necessary universal hardware platform are preferred. Based on such an understanding, the technical solutions of the present disclosure essentially, or the part contributing to the prior art may be implemented in a form of a software product. The computer software product is stored in a storage medium (such as a ROM/RAM, a magnetic disk, or an optical disc) and includes several instructions for instructing a terminal device (which may be a mobile phone, a computer, a server, a network device, or the like) to perform the methods described in the embodiments of the present disclosure.

While the particular disclosure has been described with reference to illustrative embodiments, this description is not meant to be limiting. Various modifications of the illustrative embodiments and additional embodiments of the invention will be apparent to one of ordinary skill in the art from this description. Those skilled in the art will readily recognize that these and various other modifications can be made to the exemplary embodiments, illustrated and described herein, without departing from the spirit and scope of the present invention. It is therefore contemplated that the appended claims will cover any such modifications and alternate embodiments. Certain proportions within the illustrations may be exaggerated, while other proportions may be minimized. Accordingly, the disclosure and the figures are to be regarded as illustrative rather than restrictive.

What is claimed is:

1. A device for injecting a fluid into a tissue, the device comprising:
a shaft having an elongated lumen configured to deliver a fluid from a proximal end of the shaft to a distal end of the shaft;
a handle fixed with the shaft, the handle configured to maneuver the distal end of the shaft at a tissue; and
a connector at the proximal end of the shaft, the connector configured to receive a liquid handling device.

2. The device according to claim 1, wherein:
the shaft is thinner and longer than a sheath of a hysteroscope, and the shaft is configured to feed through the sheath of the hysteroscope.

3. The device according to claim 2, wherein:
the sheath of the hysteroscope comprises an operative channel; and
an outer diameter of the shaft is smaller than an inner diameter of the operative channel, and the shaft is configured to feed through the operative channel of the hysteroscope.

4. The device according to claim 2, wherein:
the shaft is longer than the sheath of the hysteroscope along a longitudinal axis, so that when the shaft feeds through the sheath, the distal end of the shaft extends outside the sheath distally and the proximal end of the shaft extends outside the sheath proximally.

5. The device according to claim 1, wherein:
the tissue comprises a fibroid; and
the liquid comprises a vasoconstrictor reagent.

6. The device according to claim 5, wherein:
the vasoconstrictor reagent comprises vasopressin.

7. The device according to claim 1, wherein:
the distal end of the shaft comprises a beveled end configured to pierce an outer layer of the tissue.

8. The device according to claim 1, wherein:
the shaft comprises a rigid material with an outer diameter between 0.9 mm and 2.2 mm, inclusive.

9. The device according to claim 1, wherein:
the handle is configured to be operated by a health care provider, so as to maneuver the distal end of the shaft to pierce an outer layer of the tissue to deliver the liquid into the tissue.

10. The device according to claim 1, wherein:
the connector at the proximal end of the shaft comprises a female luer lock connector.

11. The device according to claim 10, wherein:
the liquid handling device comprises a syringe, wherein the female luer lock connector at the proximal end of the shaft is configured to connect to a male luer lock connector of the syringe.

12. The device according to claim 10, wherein:
the liquid handling device comprises a flexible tube and a syringe, wherein:
the female luer lock connector at the proximal end of the shaft is configured to connect to a male luer lock connector at a distal end of the flexible tube, and
a proximal end of the flexible tube is configured to connect to the syringe.

13. The device according to claim 12, wherein:
the proximal end of the flexible tube comprises a female luer lock connector; and
the female luer lock connector at the proximal end of the flexible tube is configured to connect to a male luer lock connector of the syringe.

14. The device according to claim 1, wherein:
the liquid handling device comprises a stopcock valve.

15. A system for injecting a fluid into a tissue, the system comprising:
a device comprising:
a shaft having an elongated lumen configured to deliver a fluid from a proximal end of the shaft to a distal end of the shaft;
a handle fixed with the shaft, the handle configured to maneuver the distal end of the shaft at a tissue; and
a connector at the proximal end of the shaft, the connector configured to receive a liquid handling device.

16. The system according to claim 15, wherein:
the shaft is thinner and longer than a sheath of a hysteroscope, and the shaft is configured to feed through the sheath of the hysteroscope.

17. The system according to claim 16, wherein:
the sheath of the hysteroscope comprises an operative channel; and
an outer diameter of the shaft is smaller than an inner diameter of the operative channel, and the shaft is configured to feed through the operative channel of the hysteroscope.

18. The system according to claim 16, wherein:
the shaft is longer than the sheath of the hysteroscope along a longitudinal axis, so that when the shaft feeds through the sheath, the distal end of the shaft extends outside the sheath distally and the proximal end of the shaft extends outside the sheath proximally.

19. The system according to claim 15, wherein:
the shaft comprises a rigid material with an outer diameter between 0.9 mm and 2.2 mm, inclusive.

20. A method for manufacturing a device for injecting a fluid into a tissue, the method comprising:
obtaining a shaft having an elongated lumen configured to deliver a fluid from a proximal end of the shaft to a distal end of the shaft;
fixing a handle with the shaft, the handle configured to maneuver the distal end of the shaft at a tissue; and
disposing a connector at the proximal end of the shaft, the connector configured to receive a liquid handling device.
